(19) United States
(12) Patent Application Publication
(10) Pub. No.: US 2020/0020443 A1
(43) Pub. Date: Jan. 16, 2020

(54) REMOTE MONITORING OF EQUIPMENT ASSOCIATED WITH RENAL TREATMENTS

(71) Applicant: Fresenius Medical Care Holdings, Inc., Waltham, MA (US)

(72) Inventors: Jay D. Nielsen, Castle Rock, CO (US); Thomas L. Stewart, Lakewood, CO (US); Jonathan E. Cox, Englewood, CO (US); Tobin R. Wahlers, Broomfield, CO (US)

(21) Appl. No.: 16/031,194

(22) Filed: Jul. 10, 2018

(51) Int. Cl. G16H 40/67; G16H 20/17

(52) U.S. Cl. CPC G16H 40/67; A61M 1/1601; G16H 20/17

(57) ABSTRACT

Remote monitoring of operating parameters of equipment associated with renal treatments (e.g., hemodialysis and/or peritoneal dialysis) may include using a processor and computer-readable medium operably connected to the equipment and capable of receiving and storing data related to the operating parameters of the equipment. The system may be configured to: translate the received data related to the operating parameters of the equipment; determine a risk of malfunction of the equipment based on a comparison of the translated data for a respective operating parameter of the equipment against a predetermined limit for the respective operating parameter; and generate a report indicating the operating parameters and the determined malfunction risks of the equipment. The equipment may be remotely accessible, e.g. over a network, for monitoring of the data related to the operating parameters. The equipment may include one or more refrigerators, and the system may provide for networked monitoring of multiple refrigerators.

### FIELD OF THE DISCLOSURE

The disclosure generally relates to systems and methods for monitoring equipment, and more particularly systems and methods for remote automated monitoring of equipment associated with renal (e.g., dialysis) treatments, such as refrigeration equipment, for purposes of mitigating inventory losses and/or improving personnel efficiency, among other potential benefits.

### BACKGROUND

Dialysis machines are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate or dialysis solution. The membranous lining of the patient's peritoneum acts as a natural semi-permeable membrane that allows diffusion and osmosis exchanges to take place between the solution and the blood stream. Automated peritoneal dialysis machines, called PD cyclers, may be designed to control the entire peritoneal dialysis process so that it can be performed at home, usually overnight, without clinical staff in attendance.

Some patients may administer in-home peritoneal and/or hemodialysis treatments, or may receive these treatments at out-patient facilities such as clinics, hospitals, and/or doctor's offices, and may administer or receive as part of their treatment one or more medications and/or vaccines. The medications and/or vaccines may require refrigeration for storage prior to administration to ensure safety and maintain efficacy for distribution to patient. The patient or facility may store the medications and/or vaccines in industrial, medical-grade refrigerators to prevent spoilage. An in-home or out-patient facility's equipment may be subject to regulatory requirements and/or other quality requirements, e.g., verification of temperature management of medication storage, which is typically manually verified. However, manual verification may be time consuming and inefficient, and may also allow for human error. Additionally, medical professionals and/or other caregivers may not become aware of an equipment failure until the medications and/or vaccines are no long usable, e.g., spoiled, which may result in unnecessary waste, added costs, as well as a possibility of patients not receiving an appropriate dosage and/or timing of medication.

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

An exemplary embodiment of a system for automated remote monitoring of operating parameters for equipment associated with renal treatments in accordance with the present disclosure may include a processor operably connectable to a port of the equipment, and a non-transitory computer-readable medium operably connected to the processor. The non-transitory computer-readable medium may be capable of receiving and storing data related to the operating parameters of the equipment. The non-transitory computer-readable medium may be configured to translate the received data related to the operating parameters of the equipment, determine a risk of malfunction of the equipment based on a comparison of the translated data for a respective operating parameter of the equipment against a predetermined limit for the respective operating parameter, and generate a report indicating the operating parameters and the determined malfunction risks of the equipment. The non-transitory computer readable medium may be remotely accessible for monitoring of the data related to the operating parameters.

An exemplary embodiment for a method for automated remote monitoring of equipment associated with renal treatments in accordance with the present disclosure may include operating the equipment according to one or more operating parameters. The method may include receiving data related to the operating parameters from the equipment to a non-transitory computer-readable medium operably connected to the equipment. The data may be storable in the non-transitory computer-readable medium. The method may further include translating the data related to the operating parameters of the equipment, determining a risk of malfunction of the one or more refrigerators based on a comparison of the translated data for a respective operating parameter of the equipment against a predetermined limit for the respective operating parameter, and generating a report indicating the operating parameters and the determined malfunction risks of the one or more refrigerators. The non-transitory computer-readable medium may be remotely accessible for monitoring of the data related to the operating parameters.

According to various of the foregoing and other embodiments of the present disclosure, the system may include the equipment. The generated report may be useable for mitigating inventory losses, or improving personnel efficiency, or both. The equipment may include one or more refrigerators. The operating parameters of the one or more refrigerators may include an operating temperature, a compressor temperature, a battery voltage, or a sensor, or combinations thereof. The sensor may include a door closure sensor, such that in response to an improper closure of a door of the one or more refrigerators, the system may be configured to trigger an alert off of a signal received from the sensor. In response to a decrease in the battery voltage, the system may be configured to trigger an alert. The non-transitory computer-readable medium may be configured to determine the risk of malfunction of the equipment based on a comparison against a maximum limit, a minimum warning limit, a maximum warning limit, or combinations thereof, of the operating parameters. In response to the determined malfunction risk being based on the comparison resulting in a limit value for the operating parameters exceeding the maximum warning limit, falling below the minimum warning limit, or both, the system may be configured to trigger a warning. In response to the determined malfunction risk being based on the comparison resulting in a limit value for the operating parameters exceeding the maximum limit, falling below the warning limit, or both, the system may be configured to trigger an alarm. The data may be continuously receivable to the non-transitory computer-readable medium. The data maybe continuously receivable to the non-transitory computer-readable medium in real-time. The generated report may include a record log. The system may include a server operably connected to the non-transitory computer-readable medium, and may be configured to receive the report over a network. The system may include a network communication unit that enables data communication with the non-transitory computer readable medium over a network.

### BRIEF DESCRIPTION OF THE DRAWINGS

By way of example, specific embodiments of the disclosed methods and devices will now be described, with reference to the accompanying drawings, in which:

FIGS. 1A-1C are schematics illustrating exemplary embodiments of systems for remote automated monitoring of refrigeration equipment in accordance with the present disclosure;

FIGS. 2A-2C illustrate exemplary embodiments of charting of operating parameters by remotely monitoring refrigeration equipment in accordance with the present disclosure;

FIG. 3 is a schematic illustrating an exemplary embodiment of a network system that may be used and operate in accordance with the present disclosure;

FIG. 4 illustrates an exemplary embodiment of data related to operating parameters by remotely monitoring refrigeration equipment in accordance with the present disclosure; and

FIG. 5 illustrates an exemplary embodiment of data related to operating parameters by remotely monitoring refrigeration equipment in accordance with the present disclosure.

### DETAILED DESCRIPTION

The present disclosure will now be described more fully hereinafter with reference to the accompanying drawings, in which several exemplary embodiments are shown. The subject matter of the present disclosure, however, may be embodied in many different forms and types of methods and devices and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

Systems and methods in accordance with the present disclosure may allow for automated remote monitoring of operating parameters of equipment, e.g., medical grade refrigerators. In some embodiments, the refrigerator may be configured for automated remote control of operating parameters, e.g., changing an operating temperature, turning on and/or shutting down the refrigerator, locking out the refrigerator operating parameters, and/or trigger visual warning on the refrigerator. In some embodiments, a refrigerator may be a Helmer refrigerator.

Although a refrigerator is described throughout the description as operable equipment associated with a renal treatment, such as dialysis, it is envisioned that any type of machine or equipment may be configured in accordance with the systems and methods described herein, as operable equipment in a system that is associated with a particular treatment where automated monitoring of strict operating parameters is important. For example, one or more refrigerators, or other equipment associated with medical treatment such as dialysis machines or other clinic machines, may be operably connected in a network for continuous monitoring and proactively addressing potential malfunctions, or mechanical failures.

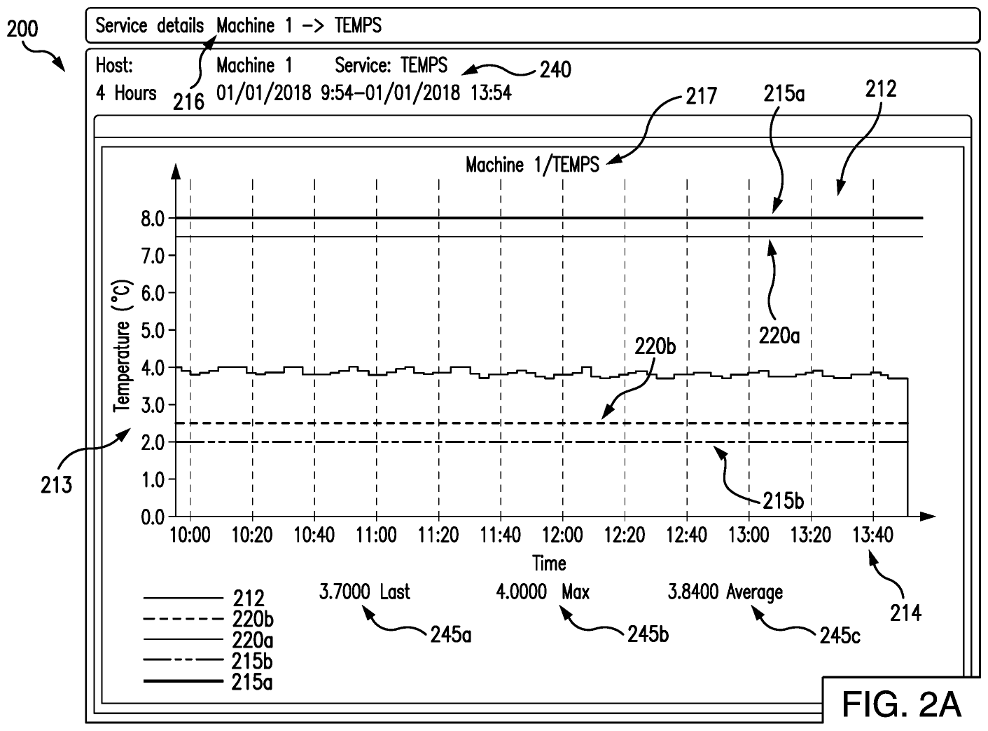

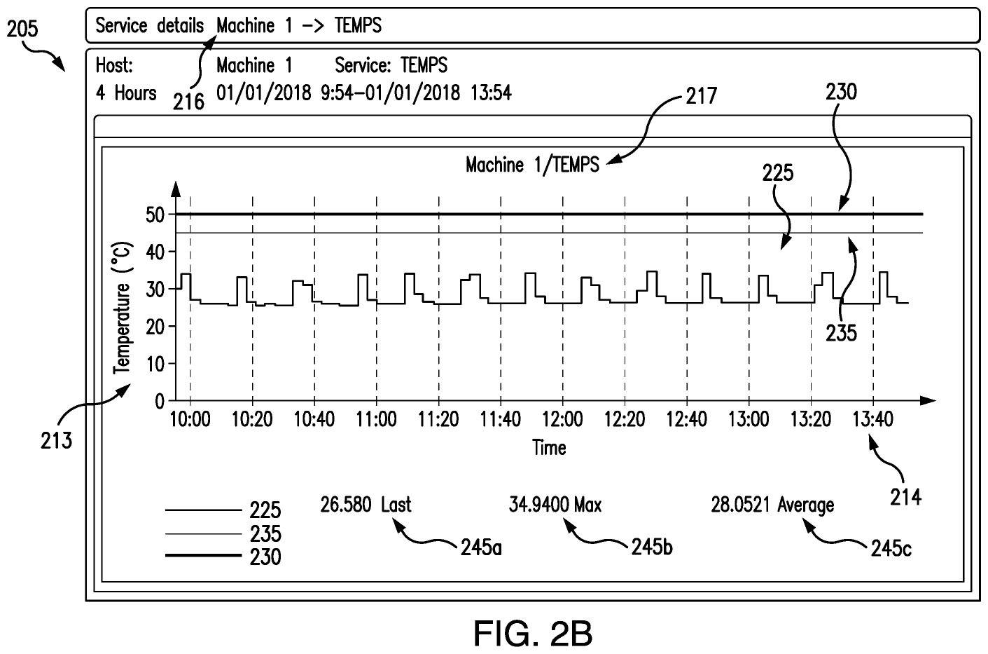

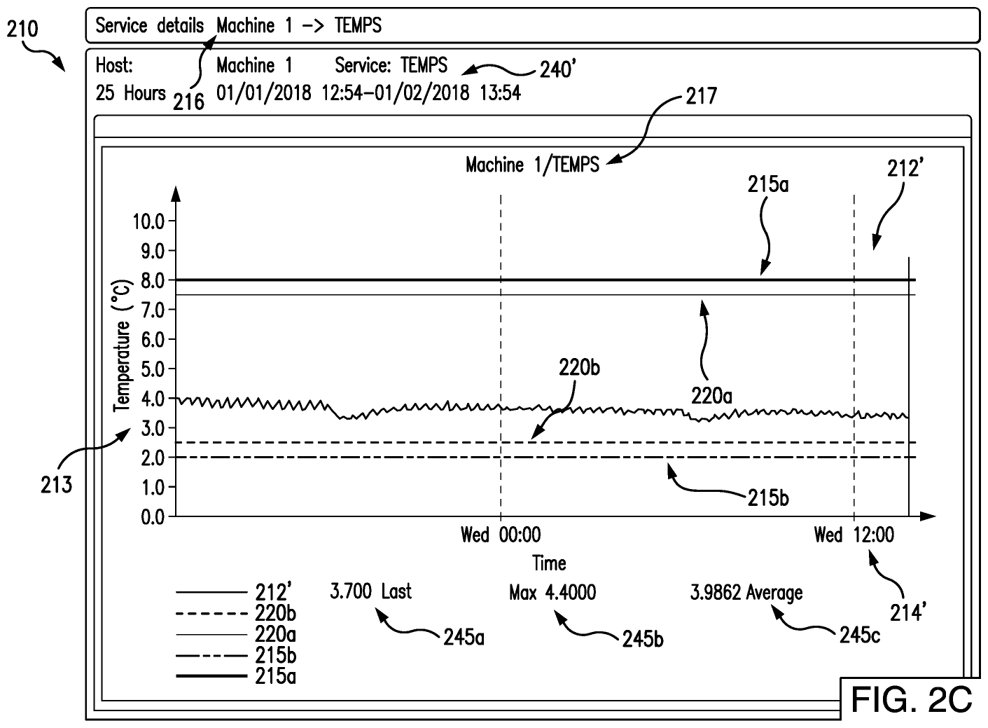

A networked system as described herein may mitigate inventory losses, and/or improve efficiency of clinic personnel through automated remote monitoring of operating parameters of the refrigerator. Additionally, systems and methods in accordance with the present disclosure may streamline regulatory design, validation, verification and/or maintenance requirements. In embodiments, the automated remote monitoring may be continuous and in real-time, so that the refrigerator is monitored around the clock, regardless of whether a facility is open for receiving patients. A report (see FIGS. 2A-2C, 4-5) may be generated and/or otherwise made accessible to various parties, including facility personnel and information technology or network personnel, which may include alerts, including warnings and/or alarms indicating unacceptable operating parameters, thereby initiating response protocols.

As described above, medical grade refrigerators associated with renal treatments, e.g., dialysis clinics, may be used for storing medications and/or vaccines within a specified threshold temperature range. For example, the refrigerators may be set to maintain an operating temperature between approximately 0° C. to 10° C., or 2° C. to 8° C., to prevent medications and/or vaccines from spoiling or otherwise becoming unusable. In some embodiments, the operating temperature may be set to the average temperature between the maximum and minimum thresholds. Regulatory requirements may dictate record management for verifying that medications and/or vaccines are maintained within a threshold temperature range, thereby requiring a written record of the temperature of the refrigerator in which they are stored. Record management policies may also have retention requirements, such as format (e.g., hard copies, electronic copies, etc.), and/or timing (e.g., how long records must be kept on premises). Operating parameters, e.g., including operating temperature data, of refrigerators may be monitored remotely and continuously, e.g., automatically, in accordance with exemplary embodiments of the present disclosure as described herein.

The refrigerator may be located in a facility that is not staffed 24 hours per day. Some facilities may only operate a few days per week and/or for a few hours at a time. As such, personnel may be unable to manually monitor the operating parameters of the refrigerator. If the refrigerator malfunctions and/or becomes otherwise inoperable, personnel may be unaware for several days, which may be too late to mitigate losing inventory of vaccines and/or medications stored in the refrigerator. Remote monitoring of refrigerator operating parameters may therefore provide continuous monitoring and may alert a user of a potential or risk of malfunction or other maintenance need of the refrigerator, which may reduce lost inventory costs. In some embodiments, the continuous monitoring may be provided in real-time. Additionally, when a facility is open for operation, remote monitoring of the refrigerator may improve personnel efficiency, improve productivity costs, decrease cost of labor, by reducing, or eliminating, manual monitoring. For example, instead of a user in each facility manually monitoring each refrigerator, remote monitoring may allow for a single user to verify a plurality of refrigerators in multiple facilities, in any geographic location, and may alert a single individual to a refrigerator malfunction, e.g., an information technology, or network, facilitator. The facilitator may then be able to inform appropriate facility personnel for additional analysis and further action as necessary. For example, an information technology or network facilitator may receive an alert indicating a refrigerator may have an operating parameter outside of an acceptable operating threshold range.

In some embodiments, the information technology or network facilitator may contact facility personnel for analysis and follow-up with the refrigerator. In some embodiments, the system may automatically initiate a correspondence to the facility personnel, e.g., an automated phone call, e-mail, or other signal, for indicating that a refrigerator may require a physical check. For example, facility personnel may have greater familiarity with the refrigerator and its particular contents. If a refrigerator has limited contents, personnel may take no immediate action, and address refrigerator maintenance according to standard procedures. If a refrigerator includes vaccines and/or medications that are critical, personnel may be able to schedule emergency maintenance on the malfunctioning machine, move the contents to an alternative location, and/or purchase another refrigerator, to minimize spoilage.

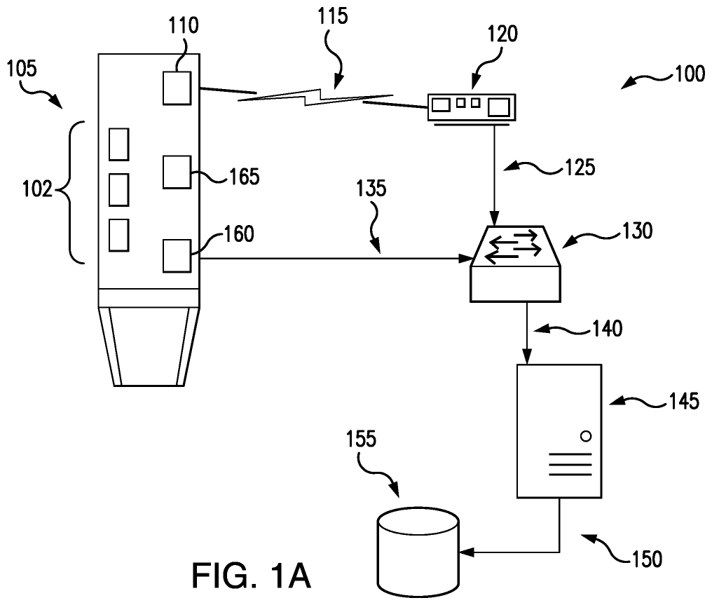

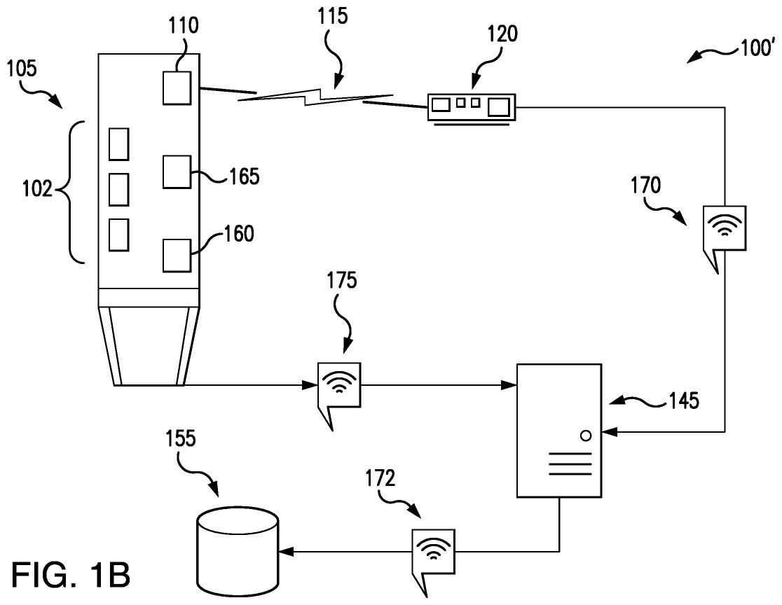

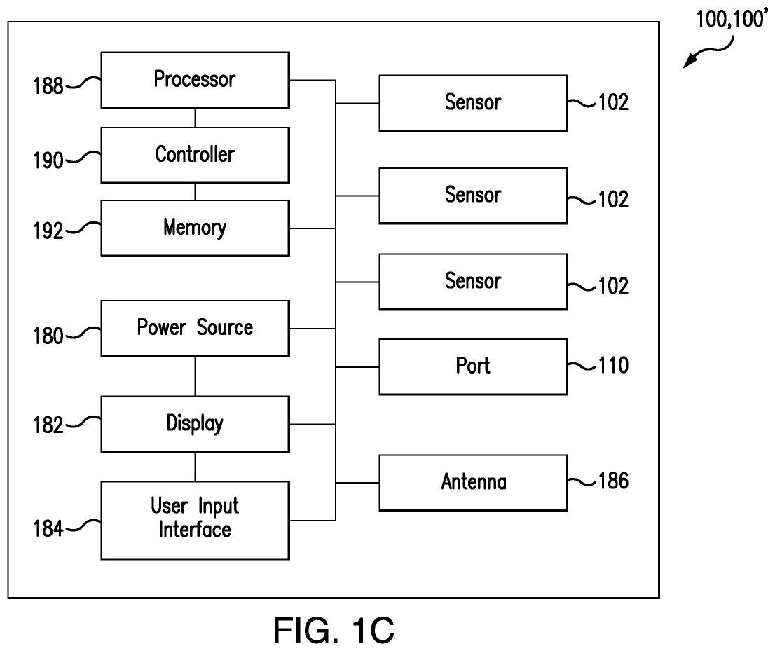

Referring now to FIGS. 1A-1C, exemplary embodiments of a system 100, 100′ in accordance with the present disclosure is shown. Although the schematics illustrate features of the system 100, 100′ as separate and individual, it is understood that any combination of the features may be integral with each other. As shown in FIGS. 1A-1C, one or more machines or equipment, e.g., a refrigerator 105, may include a port 110. In some embodiments, the system 100, 100′ may include a processor 188, a controller 190, and/or a memory 192, for storing operating parameters of the system 100, 100′. The system 100, 100′ may also include a display 182, e.g., for displaying one or more operating parameters, and/or a user input interface 184, which may allow for a user to manually change one or more operating parameters.

In some embodiments, the port 110 may be configured to operably connect RS232, RS485, RS485, or RS422 serial ports, or other standard protocols, for transmitting data. In other embodiments, the port 110 may be a universal serial bus (USB) port and/or other type of data transmission or exchange port or Ethernet. It is understood that data signals from any protocol may be transmittable from the refrigerator 105. As indicated at reference numeral 115, data may be transmittable from the refrigerator 105 to an ethernet converter 120. In some embodiments, the connection 115 may be a wired and/or wireless connection to the ethernet converter 120. In some embodiments, the ethernet converter 120 may be included in the system 100 for converting RS232 (or other recommended standard “RS” protocols) serial data signals to ethernet IP/TCP information packets, or vice versa. In other embodiments, the refrigerator 105 may directly provide Ethernet connectivity such that an ethernet converter 120 is unnecessary.

In some embodiments, e.g., in a wired system configuration (see FIG. 1A), as indicated at reference numeral 125, the converted data may be transmittable to a communication unit 130, for example, from the ethernet converter 120. In some embodiments, data signals may be transmittable from the refrigerator 105 directly to the communication unit 130, as indicated at reference numeral 135. In some embodiments, the ethernet converter 120 and the communication unit 130 may be integrated into the same unit. The converted data signals transmittable at reference numerals 125 and/or 135 may be a standard ethernet protocol such as IEEE 802.3 and/or other appropriate network protocols. Data signals may be transmittable from the communication unit 130 to a monitoring device 145 over a communication channel 140.

In some embodiments, the monitoring device 145 may be a non-transitory computer-readable medium, a storage device, laptop or desktop device, and/or a server, which may include a memory, processor, controller, display, and/or user input interface.

In some embodiments, the monitoring device 145 may be located locally to the communication unit 130, and the communication channel 140 may be a wired connection and/or a connection over a local area network. The monitoring device 145 may then be communicably coupled to a remote monitoring site, processor and/or database, represented by element 155, with enabled remote communication over a network communication channel 150, such as an Internet connection. Accordingly, in some embodiments, the monitoring device 145 may process the data of the refrigerator 105 locally and transmit a process signal remotely to the monitoring site 155 for further analysis and report generation.

In some embodiments, the communication unit 130 may include a network gateway device and the communication channel 140 may be a network connection, such as an Internet connection, as further discussed in detail elsewhere herein. Accordingly, in some embodiments, the communication unit 130 may transmit the refrigerator data to the remote monitoring device 155 which may then process and analyze the refrigerator data remotely.

FIG. 1B illustrates a wireless system configuration, e.g., data signals may be transmittable from the ethernet converter 120 to the monitoring device 145 wirelessly as indicated at reference numeral 170. In some embodiments, the refrigerator 105 may be configured to send data signals to the monitoring device 145 wirelessly as indicated by reference numeral 175, e.g., by an antenna 186. In some embodiments, the data signals may be wirelessly transmittable in accordance with standard protocols such as IEEE 802.11 over a local area network to the monitoring device 145. In other embodiments, the communications elements 170 and/or 175 may represent mobile telecommunication components that enable data signal transmission remotely to the monitoring device 145 using mobile telecommunication networks and protocols. In some embodiments, the monitoring device 145 may be local to the refrigerator 105 and include wireless communication elements, including mobile telecommunication elements to enable remote communication with the remote monitoring device 155 over a mobile telecommunication channel 172.

The monitoring device 145 may be configured to translate the data signals received from refrigerator 105, ethernet converter 120, and/or communication unit 130, into a readable format. For example, temperature data may be displayable as a chart and/or graph and may be at least a portion of a generated report (see FIGS. 2A-2C, 4-5). The monitoring device 145 may also analyze the data for determining a risk of equipment malfunction and/or failure. In some embodiments, monitoring device 145 may have one or more databases, programs, and/or algorithms for determining if a refrigerator 105 is malfunctioning, trending toward a malfunction, and/or has failed.

By sending the data from the refrigerator 105 to the monitoring device 145, the system 100 may remotely monitor one or more operating parameters of the refrigerator 105. In some embodiments, operating parameters of a refrigerator may include operating temperatures, compressor temperatures, and/or a battery voltage. In some embodiments, one or more sensors 102 may be included in the refrigerator for measuring operating parameters. For example, sensors 102 may be one or more temperature sensors, voltage sensors, and/or door closure sensors, and may be included in the system 100.

The temperature sensors may detect an operating temperature of the refrigerator, for example, as described above. In some embodiments, a temperature sensor may be operably connected to a compressor 165 of the refrigerator 105. For example, a compressor 165 may begin to malfunction or otherwise become inoperable during operation, which may result in the compressor 165 becoming heated above normal operating temperatures. This increase in compressor temperatures may indicate an impending increase in the operating temperatures, in that as the compressor 165 malfunctions, the refrigerator operating temperatures (e.g., the threshold temperature range) may not be maintainable. As described above, an information technology or network facilitator may receive an alert on a compressor indicating that the compressor temperature may be outside an acceptable operating threshold range. In some embodiments, the information technology or network facilitator may contact facility personnel for awareness and follow-up with the refrigerator.

A voltage sensor, or voltage detector, may be operably connected to a battery 160 in the refrigerator. The battery 160 may power components of the system 100, 100′ (e.g., refrigerator 105), for example, the display 182 and/or the user input interface 184, for such purposes as to allow a user to open the refrigerator 105 in the event of a power disruption. The battery 160 may also enable data collection of the refrigerator 105 throughout the power disruption. In some embodiments, during normal operation of the refrigerator 105, in response to a battery charging disruption (e.g., charging failure) and/or a battery failure, a decrease in voltage may be detectable by the voltage detector. In some embodiments, the system 100, 100′ may include a generator (not shown), so that it may be determinable if the battery 160 is discharging at a faster rate than a predetermined acceptable rate, which may also indicate that the battery 160 should be replaced. The voltage sensor may detect when more power is drawn on the battery 160, which may result in a decrease in voltage. When the voltage decreases by more than a predetermined amount, e.g., the voltage drops below a minimum threshold, it may indicate a maintenance need of the refrigerator 105. As described above, an information technology or network facilitator may receive an alert on voltage indicating the battery voltage is below an acceptable threshold. In some embodiments, the information technology or network facilitator may contact facility personnel for awareness and follow-up with the refrigerator.

In some embodiments, a door closure sensor may be included for detecting whether a door of the refrigerator has been properly closed. If the refrigerator door has been inadvertently left open by clinic personnel, or a door seal is compromised, the threshold temperature range may not be maintainable, therefore risking the refrigerator contents (e.g., medications and/or vaccines). The door closure sensor may alert a user to the refrigerator door status before contents of the refrigerator are spoiled, and may initiate response protocols for mitigating potential inventory losses. For example, as described above, an information technology or network facilitator may receive an alert on a door sensor indicating a refrigerator door is opened at a facility. In some embodiments, the information technology or network facilitator may contact facility personnel for awareness and follow-up with the refrigerator.

In some embodiments, the monitoring device 145 may have predetermined limits for each of the operating parameters. For example, operating temperatures and/or compressor temperatures of the refrigerator 105, or a battery voltage may have predetermined threshold values that may be stored in a database, or may be entered by a user, or both. A maximum limit and/or a minimum limit may be applied to the data, for verifying the data is within a predetermined boundary. In some embodiments, the monitoring device 145 may include additional predetermined limits, for example, for issuing one or more warnings prior to exceeding an acceptable temperature, or other value. In some embodiments, the monitoring device 145 may monitor data trends of the operating parameters. For example, a predetermined value may be set and include an acceptable range of the operating parameters, a standard deviation, or a percentage variation from the predetermined value. The monitoring device 145 may be configured to trigger an alert in response to an increase and/or decrease in the data, e.g., based on the trending data.

The monitoring device 145 may receive the operating parameters from the refrigerator 105, e.g., as described above with respect to FIGS. 1A-1C. As described above, data including the operating parameters may be receivable from the refrigerator 105 in any format. The monitoring device 145 may convert, or translate, the data related to the operating parameters to a readable format. When the received data has been translated, the monitoring device 145 may determine if the data is within predetermined threshold values.

Referring now to FIGS. 2A-2C, charts 200, 205, and 210 illustrate data received from the refrigerator 105 by the monitoring device 145 in a readable format for a report, and for determining an operation status of the refrigerator 105, and a potential risk of malfunction and/or failure. As shown in FIG. 2A, the chart 200 may illustrate an operating temperature 212 of the refrigerator 105 for a refrigerator indicated at reference numeral 216. The operating parameter associated with the chart may be indicated at reference numeral 217, e.g., “temps” for an operating temperature, or a compressor temperature, or both, as a category of the monitored operating parameter. It is also understood that charts 200, 205, 210 may illustrate data related to operating parameters for battery voltage as well as other sensor indicators, including but not limited to a door sensor.

In some embodiments, an acceptable operating temperature threshold range may be between 0° C. and 10° C., or 2° C. and 8° C., as indicated at reference numeral 213 as the y-axis. Although these temperature ranges are described herein, it is understood that the refrigerator 105 may be set to operate within any set temperature range, e.g., between approximately −10° C. and 20° C. A maximum limit of 8° C. may therefore be set, as indicated at reference numeral 215a, and a minimum limit of 2° C. may be set as indicated at reference numeral 215b. If the refrigerator is operating at temperatures above 8° C. or below 2° C., the monitoring device may generate an alarm so that a user may be made aware of the malfunction to take action to mitigate or prevent spoilage of vaccines and/or medications stored in the refrigerator 105.

In some embodiments, additional predetermined limits may be included to generate an alert to a user before the refrigerator reaches unacceptable temperatures. For example, an additional predetermined limit may be set as 0.5 degrees within the maximum limit and/or minimum limit. In some embodiments, the additional predetermined limit may be set as any temperature within the operating temperature threshold, e.g., within approximately 0.1 degrees and 2 degrees. For example, when a maximum limit is set as 8° C., an additional predetermined maximum limit may be set at 7.5° C. as indicated at reference numeral 220a, and when a minimum limit is set as 2° C., an additional predetermined minimum limit may be set as 2.5° C. as indicated at reference numeral 220b. In this manner, if the refrigerator 105 is operating at a temperature setpoint of approximately 4° C. and begins to drift toward the limits 215a, 215b, an additional limit 220a, 220b may provide warning to a user to proactively provide machine maintenance and/or move vaccines and/or medications to an alternative location prior to exceeding the acceptable threshold limit, thereby mitigating inventory losses being stored in the refrigerator 105.

Referring now to FIG. 2B, a chart 205 may illustrate a compressor temperature 225 of the refrigerator 105. As described above, a temperature of the compressor may provide early signs to a user that a refrigerator may require maintenance. As described above with respect to FIG. 2A, the monitoring device 145 may determine if the compressor temperature is operating within an acceptable temperature range, e.g., by a predetermined maximum limit as indicated by reference numerals 230. In some embodiments, an additional predetermined maximum limit may be included as a temperature within the maximum limit, to provide a warning to a user before a temperature exceeds the set threshold. For example, as shown in reference numeral 235, an additional predetermined maximum limit may be set so that if the compressor temperature 225 drifts towards the maximum limit 230, the additional limit 235 may provide warning to a user to proactively provide machine maintenance and/or relocate contents of the refrigerator 105.

In some embodiments, the charts 205, 210 may illustrate different operating parameters for the same refrigerator 105, over the same period of time. In some embodiments, the charts 205, 210 may illustrate any operating parameter of the refrigerator 105, and may illustrate different time periods. For example, the charts 200, 205, 210, may show any time range of operating parameters of the refrigerator 105, as indicated at reference numeral 214 as the x-axis. As shown in FIGS. 2A-2B, temperature monitoring over a four-hour period of time is illustrated, as indicated at reference numeral 240 respectively. An advantage to continuous monitoring of the refrigerator 105 may allow for analysis of an operating parameter over any period of time. A particular time range may be required for record keeping purposes to satisfy regulatory requirements. Referring now to FIG. 2C, a chart 210 may illustrate an operating temperature 212′ of the refrigerator 105. FIG. 2C may differ from FIG. 2A only in that a monitored time period may be an extended period of time, e.g., 25 hours, as indicated at reference numeral 240′. The monitoring device 145 may have predetermined time ranges for generating charts 200, 205, 210. In some embodiments, a user may enter a unique time range for generating a report. It may be advantageous to generate charts including different time periods for a refrigerator 105, as the monitoring device 145 may be able to determine longer-term trends in the refrigerator 105. Additionally, as described above, as some facilities may be closed for several days at a time, leaving the refrigerator 105 entirely unsupervised, a user may rely on charts including extended time periods for verifying a consistency in operating parameters when the facility is closed, or inconsistency in operating parameters that is of concern, but corrects itself prior to the facility opening (e.g., prolonged power outage).

The charts 200, 205, 210 may also include additional parameters 245a, 245b, . . . 245n, where “n” may be any number of additional parameters. For example, as shown in FIGS. 2A-2C, additional parameters 245a-245c may include a most recent, or last, operating temperature, a maximum temperature measured over the predetermined time period 240, 240′, and an average temperature measured over the predetermined time period 240, 240′. It is understood that the additional parameters 245a, 245b, . . . 245n may be any parameter determinable from the operating parameters associated with the respective chart 200, 205, 210, including but not limited to maximum temperatures, minimum temperatures, average temperatures, standard deviation, voltage, and other sensor data.

Medical devices, equipment or associated peripherals may be provided with functionality to connect through a secure gateway to a network, including an external network to send and receive information to a clinic or remote monitoring station. The connection, network and data transmissions among components, both local and external, may be controlled and/other otherwise incorporated into a system that facilitates such functions with appropriate network infrastructure, and which may, in some implementations, be referred to as a connected health (CH) system. For further descriptions of systems for securely connecting, pairing and/or monitoring medical devices, reference is made to U.S. Pub. No. 2016/0206800 entitled “Remote Monitoring Interface Device and Mobile Application for Medical Devices” to Tanenbaum et al., U.S. Pat. No. 9,800,663 entitled “Associating Dialysis Accessories Using Near Field Communication” to Arrizza, U.S. Pub. No. 2017/0087290 entitled “Short-Range Wireless Communication for a Dialysis System” to Medina et al., U.S. Pub. No. 2017/0076069 entitled “Secure Network-Based System for Communication of Clinical Data” to Moissl et al., and U.S. Pat. No. 9,178,891 entitled “Remote Control of Dialysis Machines” to Wang et al., the disclosures of all of which are hereby incorporated by reference in their entireties.

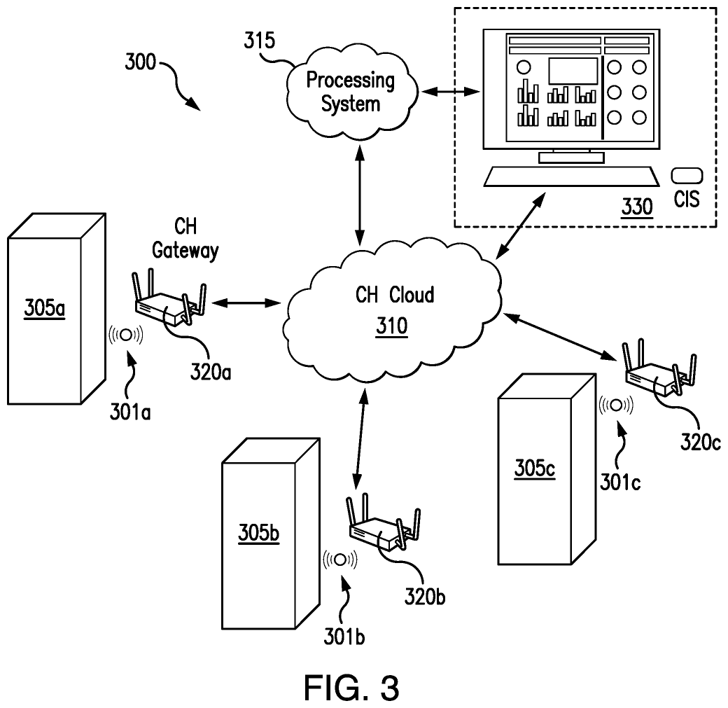

FIG. 3 is a schematic illustration showing an example of a network system, such as a connected health (CH) system 300, that may include, among other things, a CH cloud service 310, a processing system 315, and a gateway (CH Gateway), such as gateway 320a, which may be used in connection with network aspects of the systems described herein. The processing system 315 may be a server and/or cloud-based system that processes, conducts compatibility checks and/or formats medical information, including information generated at a clinical information system (CIS) 330 of a clinic or hospital or other central monitoring site, in connection with data transmission operations of the CH system 300. The CH system 300 may include appropriate encryption and data security mechanisms. The CH cloud service 310 may be a cloud-based application that serves as a communication pipeline (e.g., facilitates the transfer of data) among components of the CH system 300 via connections to a network such as the Internet. The gateway 320a may serve as a communication device facilitating communication among components of the CH system 300. In various embodiments, the gateway 320a is in communication with a refrigerator 305a; this communication may include or incorporate other signal components, such as a converter, like that discussed elsewhere herein. The communication channel may be via a wireless connection 301a, such as a Bluetooth, Wi-Fi and/or other appropriate type of local or short range wireless connection. In other embodiments, the gateway 320a may be in wired communication with the refrigerator 305a (including via one or more other signal components). The gateway 320a may also be in connection with the CH cloud service 310 via a secure network (e.g., Internet) connection. The gateway 320a may be configured to transmit/receive data to/from the CH cloud service 310 and transmit/receive data to/from the refrigerator 305a.

As further shown, the system described herein may be used with multiple refrigerators 305b, 305c, . . . 305n, which may be located at multiple remote sites, including at one or more clinics and/or in one or more patient's homes. The remotely located refrigerators 305b, 305c, . . . 305n may each operate in connection with gateways 320b, 320c, . . . 320n, using communication channels 301b, 301c, . . . 301n, like that discussed above in connection with refrigerator 305a, gateway 320a and communication channel 301a, and may communicate via the CH cloud 310 with the CIS 330 or other central monitoring station in accordance with monitoring functionality of the connected health system 300, as further discussed elsewhere herein.

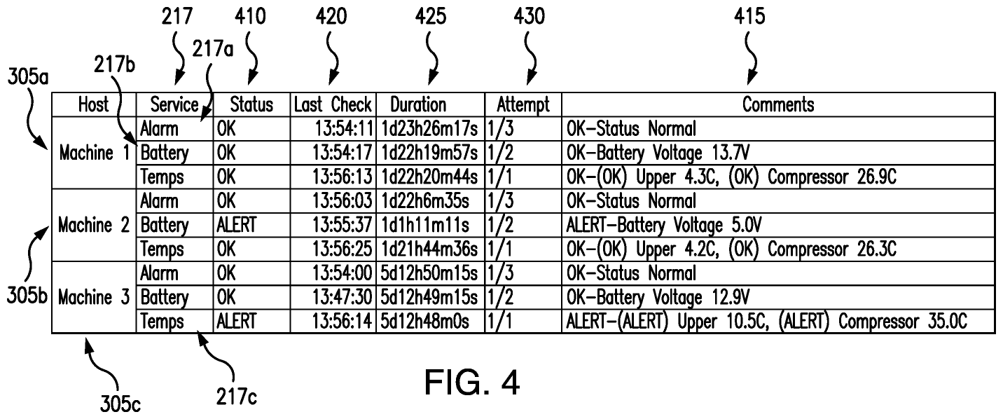

As described above, a remote user such as an information technology or network facilitator, may monitor a plurality of machines across multiple facilities geographically separated from each other. Each refrigerator may be monitored for unacceptable operating parameters (e.g., drifting outside of an acceptable operating threshold range). Referring now to FIG. 4, a report 400 may be generated, compiling a plurality of refrigerators 305, including 305a, 305b, . . . 305n, where “n” is any number of refrigerators. It is also understood that the refrigerators 305a, 305b, . . . 305n may be located in facilities in any geographic location.

For each refrigerator 305a, 305b, . . . 305n, a category 217a, 217b, . . . 217n indicating the monitored operating parameter may be included. As described above, the category may include an operating temperature, a compressor temperature, a battery voltage, a door sensor, or other sensor data. Although categories 217a-217c are illustrated in FIG. 4, it is understood that any number “n” of categories may be included in the report 400 for each refrigerator 305. The report 400 may include a summary status 410 of each category 217, e.g., indicating if the selected operating parameter is “OK” when the operating parameters are within an acceptable operating threshold, or “ALERT” when the operating parameters are outside of an acceptable operating threshold. In some embodiments, a comment section 415 may be included for each operating parameter category 217 of each refrigerator 305, which may include additional information regarding the operating parameter and/or its summary status 410. For example, one or more additional parameters 245a, 245b, . . . 245n may be output in the respective comments 415. If an operating parameter is outside of an acceptable operating temperature threshold range, the measured value of the operating parameter may be output.

In some embodiments, one or more of the refrigerators 305a, 305b . . . 305n may be configured for automated remote control of operating parameters, e.g., changing an operating temperature, turning on and/or shutting down the refrigerator, locking out the refrigerator operating parameters, and/or trigger visual warning on the refrigerator. Using one or more components of the CH system 300, information, instructions and/or commands may be sent, transmitted and/or otherwise downloaded from the CIS 330 and/or other centralized monitoring station via the CH system 300 infrastructure to one or more of the refrigerators 305a, 305b . . . 305n in response to the report 400 and/or other notifications or alerts that control or initiate the automated remote control of the one or more operating parameters.

The report 400 may further include additional information, including a time of a most recent status check 420, a duration of time between status checks 425, and a number of attempts 430. For example, the recent status check 420 may be a record of when the selected component was last queried. The duration of status checks 425 may be a record of a length of time from the last change of statuses. The number of attempts 430 may be a number of attempts before changing the status of the selected component. In some embodiments, if after a first attempt there is no detected change of the selected component, the system may forego additional attempts. In some embodiments, if after a first attempt there is a non-response or a change in response of the selected component, additional attempts may be made to validate the change and/or non-response. Any number “n” of attempts may be set for alarms. For example, as shown in FIG. 4, alarm statuses may have a plurality of attempts (e.g., 3) to minimize false positives before issuing an alert. Battery statuses may also have a plurality of attempts (e.g., 2) prior to alerting. In some embodiments, a single attempt may be set to alert a user immediately. For example, a temperature setting may be set so that an alert may be issued when a temperature threshold is crossed.

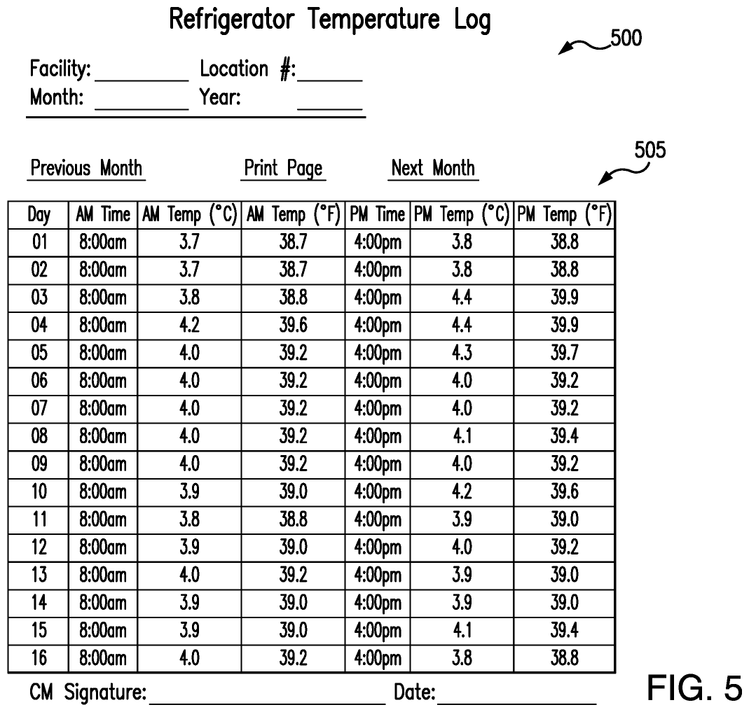

As described above, reports may be generated to satisfy regulatory requirements for a facility. As shown in FIG. 5, a report 500 may be generated indicating an operating temperature of a refrigerator 105, for predetermined selected times. For example, a discrete operating temperature of the refrigerator at selected times, e.g., 8:00 AM, and 4:00 PM, may be included as a record log 505. In some embodiments, facility personnel may be able to generate the report 500 including the record log 505 over a selected time period, e.g., on a monthly basis, for each refrigerator 105. The record log 505 may verify that the operating parameter (e.g., operating temperature) for storing vaccines and/or medications are within an acceptable range. Another advantage in having continuous, remote monitoring of the operating parameters is that facility personnel may not only verify that an operating parameter is acceptable at discrete selected times to satisfy regulatory requirements, but also may provide by continuous monitoring an additional assurance that an operating parameter has not drifted outside an acceptable operating temperature threshold range at a time other than the discrete selected times. This may be advantageous in ensuring inventory, e.g., vaccines and/or medications, may be safe and effective for distribution to patients.

Some embodiments of the disclosed systems may be implemented, for example, using a storage medium, a computer-readable medium or an article of manufacture which may store an instruction or a set of instructions that, if executed by a machine (i.e., processor or microcontroller), may cause the machine to perform a method and/or operation in accordance with embodiments of the disclosure. In addition, a server or database server may include machine readable media configured to store machine executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

To the extent used in this description and in the claims, a recitation in the general form of “at least one of [a] and [b]” should be construed as disjunctive. For example, a recitation of “at least one of [a], [b], and [c]” would include [a] alone, [b] alone, [c] alone, or any combination of [a], [b], and [c].

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A system for automated remote monitoring of operating parameters for equipment associated with renal treatments, comprising:
a processor operably connectable to a port of the equipment;
a non-transitory computer-readable medium operably connected to the processor and capable of receiving and storing data related to the operating parameters of the equipment, wherein the non-transitory computer-readable medium is configured to:
translate the received data related to the operating parameters of the equipment;
determine a risk of malfunction of the equipment based on a comparison of the translated data for a respective operating parameter of the equipment against a predetermined limit for the respective operating parameter; and
generate a report indicating the operating parameters and the determined malfunction risks of the equipment; and
wherein the non-transitory computer-readable medium is remotely accessible for monitoring of the data related to the operating parameters.

2. The system according to claim 1, further comprising the equipment.

3. The system according to claim 1, wherein the generated report is useable for mitigating inventory losses, or improving personnel efficiency, or both.

4. The system according to claim 1, wherein the equipment includes one or more refrigerators.

5. The system according to claim 4, wherein the operating parameters of the one or more refrigerators include an operating temperature, a compressor temperature, a battery voltage, or a sensor, or combinations thereof.

6. The system according to claim 5, wherein the sensor includes a door closure sensor, such that in response to an improper closure of a door of the one or more refrigerators, the system is configured to trigger an alert off of a signal received from the sensor.

7. The system according to claim 5, wherein in response to a decrease in the battery voltage, the system is configured to trigger an alert.

8. The system according to claim 1, wherein the non-transitory computer-readable medium is configured to determine the risk of malfunction of the equipment based on a comparison against a maximum limit, a minimum warning limit, a maximum warning limit, or combinations thereof, of the operating parameters.

9. The system according to claim 8, wherein in response to the determined malfunction risk being based on the comparison resulting in a limit value for the operating parameters exceeding the maximum warning limit, falling below the minimum warning limit, or both, the system is configured to trigger a warning.

10. The system according to claim 8, wherein in response to the determined malfunction risk being based on the comparison resulting in a limit value for the operating parameters exceeding the maximum limit, falling below the warning limit, or both, the system is configured to trigger an alarm.

11. The system according to claim 1, wherein the data is continuously receivable to the non-transitory computer-readable medium.

12. The system according to claim 11, wherein the data is continuously receivable to the non-transitory computer-readable medium in real-time.

13. The system according to claim 1, wherein the generated report includes a record log.

14. The system according to claim 1, further comprising a server operably connected to the non-transitory computer-readable medium and configured to receive the report over a network.

15. The system according to claim 1, further comprising a network communication unit that enables data communication with the non-transitory computer readable medium over a network.

16. A method for automated remote monitoring of equipment associated with renal treatments, comprising:
operating the equipment according to one or more operating parameters;
receiving data related to the operating parameters from the equipment to a non-transitory computer-readable medium operably connected to the equipment, the data being storable in the non-transitory computer-readable medium;
translating the data related to the operating parameters of the equipment;
determining a risk of malfunction of the equipment based on a comparison of the translated data for a respective operating parameter of the equipment against a predetermined limit for the respective operating parameter;
generating a report indicating the operating parameters and the determined malfunction risks of the equipment; and
wherein the non-transitory computer-readable medium is remotely accessible for monitoring of the data related to the operating parameters.

17. The method according to claim 16, wherein the generated report is useable for mitigating inventory losses, or improving personnel efficiency, or both.

18. The method according to claim 16, wherein the equipment includes one or more refrigerators.

19. The method according to claim 18, wherein the operating parameters of the one or more refrigerators include an operating temperature, a compressor temperature, a battery voltage, or a sensor reading, or combinations thereof.

20. The method according to claim 19, wherein the sensor includes a door closure sensor, such that in response to an improper closure of a door of the one or more refrigerators, triggering an alert off of a signal received from the sensor.

21. The method according to claim 19, wherein in response to a decrease in the battery voltage, triggering an alert.

22. The method according to claim 16, wherein the non-transitory computer-readable medium is configured to determine the risk of malfunction of the equipment based on a comparison against a maximum limit, a minimum warning limit, a maximum warning limit, or combinations thereof, of the operating parameters.

23. The method according to claim 22, wherein in response to the determined malfunction risk being based on the comparison resulting in a limit value for the operating parameters exceeding the maximum warning limit, falling below the minimum warning limit, or both, triggering a warning.

24. The method according to claim 22, wherein in response to the determined malfunction risk being based on the comparison resulting in a limit value for the operating parameters exceeding the maximum limit, falling below the warning limit, or both, the triggering an alarm.

25. The method according to claim 16, wherein the data is continuously receivable to the non-transitory computer-readable medium.

26. The method according to claim 25, wherein the data is continuously receivable to the non-transitory computer-readable medium in real-time.

27. The method according to claim 16, wherein the generated report includes a record log.

28. The method according to claim 16, wherein a server operably is connected to the non-transitory computer-readable medium, and is configured to receive the report over a network.

29. The method according to claim 16, further comprising:
configuring a network communication unit to enable data communication with the non-transitory computer readable medium over a network.
